(19) United States
(12) Patent Application Publication
(10) Pub. No.: US 2007/0031364 A1
(43) Pub. Date: Feb. 8, 2007

(54) Keratin gel and the types 2,4 and 6 of collagens of frog protein topical use or for lamina preparation use, such as dressing powdering in food and complements use and in cosmetic mask manufacturing or in solutions used in cosmetic formulation and medical/pharmaceutical

(76) Inventors: Maria De Lurdes Molarinho Velly, Sao Paulo (BR); Maria Helena Farinha Verissimo, Sao Paulo (BR)

Correspondence Address: Laurence B Bond;TraskBritt, P O Box 2550, Salt Lake City, UT, 84110, US

(21) Appl. No.: 10/577,922

(22) PCT Filed:
(86) PCT No.: PCT/BR04/00209

(30) Foreign Application Priority Data
Oct. 31, 2003 (BR) PI 03040 83-6

(51) Int. Cl. A61K 8/64; A61K 38/39

(52) U.S. Cl. 424/70.14; 424/445; 514/2

(57) ABSTRACT

is constituted by keratin gel (1) and the types 2, 4 and 6 of collagens, for topical use or for lamina preparation (2) use, such as biological dressing with the gel (1) powdered (3) for food and complements use (4), or with the mentioned gel (1) and powder (3) used in cosmetic mask manufacturing (5) or turned into solutions (6) that may be used in injectable medical/pharmaceutical formulations (7) or in products of cosmetic and aesthetic topical use (8), whose development allowed gel production for topical use or for dressing lamina preparation (wound recovering from several origins) and/or powdering in food and complements and mask manufacturing used in aesthetic areas and solutions used in formulations and medical, surgical, pharmaceutical, aesthetic, cosmetic and cosmetical areas and others.

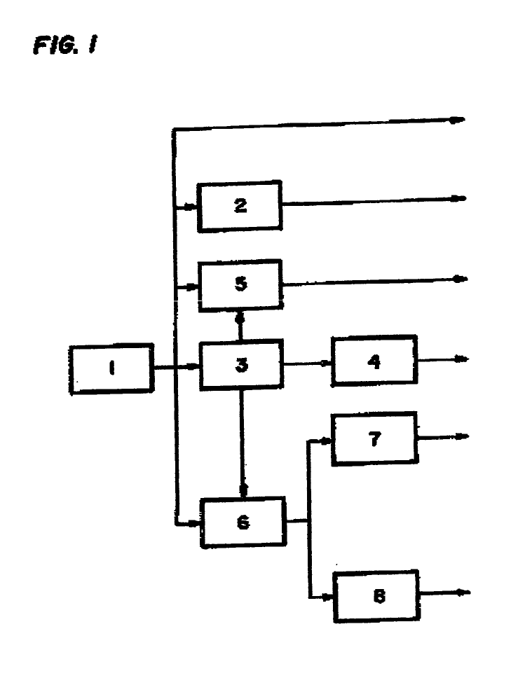

It refers, to the present descriptive report, about patent of the invention of keratin gel and the types 2, 4 and 6 of collagens of frog protein for topical use or for lamina preparation use, such as dressing with the gel powdered for food and complements use, or with the cosmetic mask manufacturing and medical/pharmaceutical formulations whose development allowed gel production for topical use or for dressing lamina preparation (wound recovering from several origins) and/or powdering in food and complements and mask manufacturing used in aesthetic areas and solutions used in formulations and medical, surgical, pharmaceutical, aesthetic, cosmetic and cosmeceutical areas and others.

It is a proved fact, the actual great society preocupation with health conservation and search increase of its vitality and consequent increase in human being's life.

This concern follows paralelly the preocupation, even bigger, of natural products search that has more effectiveness relating to the synthetic products available on the market, to assure that people could have better quality of life. Natural products with regenerating and antioxidant properties that come from aquatic environments may attend these market demands, being used pure or modified and in internal or external use and among animals from aquatic fauna, the frog has special and specific properties, once it contains a protein and enzymatic composition, among others, with very characteristical properties in each phase of its developing and cypathologic tests showed that these materials are compatible to the human being and their regenerating necessity. Keratin and the types 2, 4 and 6 of collagens are materials with high teor of Organic Silicon, with special differentiated disposition of the protein chains and with natural antibiotics, having 8 (eight) of 9 (nine) essential aminoacids, allowing them to have beneficial properties to health and may be used by medical area in general and in cosmetic and pharmaceutical area, among others.

It must, however, take care about transformation, application and/or the utilization of this protein in formulations and its benefit are made without change the natural properties of its origin. These proteins are called and defined as being the “Organic Crystals”, due to its effectiveness, as products for vitality and human life in general, had been already proved.

The petitioner requested The patent of Invention order n# PI0103866-4, that was deposited on Jul. 10, 2001 and denominated “FROG SKIN EPIDERMIS EXTRACTION PROCESS, KERATIN FILM OBTAINMENT PROCESS, POWDERED KERATIN OBTAINMENT PROCESS, KERATIN FILM, POWDERED KERATIN, KERATIN FILM USE, AND POWDERED KERATIN USE”, where protection was requested for protein material extraction process (keratin) and where it was identified the potenciality of these materials, which was defined and denominated Organic Crystal, because of its natural potenciality to health, aesthetic and organisms life and human willing and, afterwards, the types 2, 4 and 6 of collagens.

The extraction process discovery began when it was achieved to separate the epidermis of the frog's derm from type 1 collagen without using Na₂S that traditionally is used in the animal leather benefit process. It is known, that this chemical product when falls in the effluent of the tanning, it turns into H₂SO₄ and eliminates the organic substance that is the water oxygenation source, that damages excessively the quality of the aquatic fauna and, consequently, the quality of the riparian population.

The protein transformation into gel occurred through processes and traditional formulations of gel preparation, with the utilization of the elements obtained by the previous mentioned patent process.

It must be recalled that synthetic products, functionally similar to previous products are obtained through the chemical product utilization and chemical synthesizes for their preparation, presenting costs that are, in general, high and, besides, many of these synthetic products present harmful side effects to human health.

In the required patent, keratin gel and the types 2, 4 and 6 of collagens of frog protein for topical use or for lamina preparation use, such as dressing or powdering in food and complements use and in cosmetic mask manufacturing or in solutions used in cosmetic formulation and medical/pharmaceutical are constituted by the fact that this gel that may be applied in topical use, could be worked in the laminas preparation use, in medical and pharmaceutical areas, such as dressing or be turned into powder to be used in food and complements preparation or into solutions for use in cosmetic products and medical/pharmaceutical formulation with sorted concentrations.

The materials are adequately decontaminated, macerated and turned into powder 53 μM, and some are liofilizated. The destinated ones for using in food and complements are transformed into liquid and later into powder.

These proteins obtained are submitted to a chemical/physical process dilution treatment and it follows the traditional formulations for gels preparation by cosmetic, cosmeceutical, pharmaceutical and dressing areas.

The material is isolated and turned into powder by traditional methods with chemical and physical actions and the powder is diluted in proper solvents according to the material polarity to be transformed or not, into solutions to products following formulations in specific areas or similar.

The product to the biological dressing segment, surgical sector and medical sector in general, keeps the integral original composition, that presents substancial advantages, free of sequelas or scars and presenting fast cicatrisation, once it provokes the causing agents oxidation of primary infections and the fast cellular development and the injuried tissues recovery, in a very perceptible manner in the first application.

In the cosmetic and aesthetic field, the gel and the powder would be turned into masks, lotions, hair and skin products.

It must be, likewise, recall that, taking advantage of this raw material from the frog's abate, delivers the environment to receive undesirable residues.

For better understanding of this patent's subject, references will be made to the attached picture in which:

Picture 1 shows, in schematic view, a fluxogram of use and transformation of keratin gel and the the types 2, 4 and 6 of collagen of frog protein into products for direct use or like food and complement components, of products and cosmetics of topical use or injetable products for medical/pharmaceutical use.

The present patent of Invention of “KERATIN GEL THE TYPES 2, 4 AND 6 OF COLLAGENS OF FROG PROTEIN FOR TOPICAL USE OR FOR LAMINA PREPARATION USE, SUCH AS DRESSING OR POWDERING IN FOOD AND COMPLEMENTS USE AND IN COSMETIC MASK MANUFACTURING OR IN SOLUTIONS USED IN COSMETIC FORMULATION AND MEDICAL/PHARMACEUTICAL,” of keratin gel and the types 2, 4 and 6 of collagens of frog protein chemically treated, characterized by gel (1) that, properly washed and dried, may be used in lamina preparation (2) or with the mentioned gel (1) used directly for topical use and the mentioned laminas (2) used as biological dressing, with the mentioned gel (1) powdered (3), that may be used in food and complements (4), or the mentioned gel (1) and the powder (3) used for cosmetic mask manufacturing (5) or the mentioned gel (1) and the powder (3) turned into solutions (6) that will be used in injectable medical/pharmaceutical formulations (7) or in products of cosmetic and aesthetic topical use (8).

1- “KERATIN GEL AND THE TYPES 2, 4 AND 6 OF COLLAGENS OF FROG PROTEIN FOR TOPICAL USE OR FOR LAMINA PREPARATION USE, SUCH AS DRESSING OR POWDERING IN FOOD AND COMPLEMENTS USE AND IN COSMETIC MASK MANUFACTURING OR IN SOLUTIONS USED IN COSMETIC FORMULATION AND MEDICAL/PHARMACEUTICAL,” of keratin gel and the types 2, 4 and 6 of collagens of frog protein chemically treated, characterized by gel (1) that, properly washed and dried, may be used in lamina preparation (2) or with the mentioned gel (1) used directly for topical use and the mentioned laminas (2) used as biological dressing, with the mentioned gel (1) powdered (3), that may be used in food and complements (4), or the mentioned gel (1) and the powder (3) used for cosmetic mask manufacturing (5) or the mentioned gel (1) and the powder (3) turned into solutions (6) that will be used in injectable medical/pharmaceutical formulations (7) or in products of cosmetic and aesthetic topical use (8).
